(19) United States
(12) Patent Application Publication
(10) Pub. No.: US 2026/0109660 A1
(43) Pub. Date: Apr. 23, 2026

(54) METHANOL PROCESS

(71) Applicant: JOHNSON MATTHEY DAVY TECHNOLOGIES LIMITED, London (GB)

(72) Inventor: Gabriele GERMANI, Cleveland (GB)

(21) Appl. No.: 19/117,818

(22) PCT Filed: Oct. 31, 2023
(86) PCT No.: PCT/GB2023/052837

§ 371 Date: Apr. 2, 2025

(30) Foreign Application Priority Data
Nov. 16, 2022 (GB) 2217131.8

(51) Int. Cl. C07C 29/151; C07C 29/74

(52) U.S. Cl. CPC C07C 29/1516; C07C 29/74

(57) ABSTRACT

A process for the synthesis of methanol comprising the steps of: (i) passing a first synthesis gas mixture comprising a make-up gas and a loop recycle gas stream through a first synthesis reactor containing a cooled methanol synthesis catalyst to form a first product gas stream, (ii) recovering methanol from the first product gas stream thereby forming a first methanol-depleted gas mixture, (iii) passing at least a portion of the first methanol-depleted gas mixture through a second synthesis reactor containing a cooled methanol synthesis catalyst to form a second product gas stream, (iv) recovering methanol from the second product gas stream thereby forming a second methanol-depleted gas mixture, (v) passing the second methanol-depleted gas mixture through a third synthesis reactor containing a cooled methanol synthesis catalyst to form a third product gas stream, and (vi) recovering methanol from the third product gas stream thereby forming a third methanol-depleted gas mixture.

This invention relates to a process for synthesising methanol.

Methanol synthesis is generally performed by passing a synthesis gas comprising hydrogen, carbon oxides and any inert gases at an elevated temperature and pressure through one or more beds of a methanol synthesis catalyst, which is often a copper-containing composition. Methanol is generally recovered by cooling the product gas stream to below the dew point of the methanol and separating off the product as a liquid. The crude methanol is typically purified by distillation. The process is often operated in a loop: thus, the remaining unreacted gas stream is usually recycled to the synthesis reactor as part of the synthesis gas via a circulator. Fresh synthesis gas, termed make-up gas, is added to the recycled unreacted gas to form the synthesis gas stream. A purge stream is often taken from the circulating gas stream to avoid the build-up of inert gasses.

The process may be operated using multiple synthesis reactors each containing a bed of methanol synthesis catalyst.

WO2017/121980 A1 discloses a two-reactor process where both reactors are fed with a loop recycle stream, the first synthesis reactor has a higher heat transfer per cubic metre of catalyst than the second synthesis reactor and the recycle ratio to the first synthesis reactor is less than that to the second synthesis reactor.

EP3210961 A1 discloses a multiple-stage methanol synthesis process. In one embodiment, a three-stage methanol synthesis loop is depicted.

We have realised that a high-efficiency three-stage methanol synthesis unit can be realized by using a specific combination of reactors operated in a single loop with a low circulation ratio. Accordingly, the invention provides a process for the synthesis of methanol comprising the steps of:
(i) passing a first synthesis gas mixture comprising a make-up gas and a loop recycle gas stream through a first synthesis reactor containing a cooled methanol synthesis catalyst to form a first product gas stream,
(ii) recovering methanol from the first product gas stream thereby forming a first methanol-depleted gas mixture,
(iii) passing at least a portion of the first methanol-depleted gas mixture through a second synthesis reactor containing a cooled methanol synthesis catalyst to form a second product gas stream,
(iv) recovering methanol from the second product gas stream thereby forming a second methanol-depleted gas mixture,
(v) passing the second methanol-depleted gas mixture through a third synthesis reactor containing a cooled methanol synthesis catalyst to form a third product gas stream,
(vi) recovering methanol from the third product gas stream thereby forming a third methanol-depleted gas mixture; and
(vii) feeding a portion of the third methanol-depleted gas mixture to the first methanol synthesis reactor as the loop recycle gas stream,
   wherein the first and second synthesis reactors have a higher heat transfer area per cubic metre of catalyst than the third synthesis reactor, a circulating compressor is provided to compress either the first synthesis gas mixture, the first methanol-depleted gas mixture or the second methanol depleted gas mixture, and the loop recycle gas and make-up gas have a molar flow rate ratio of ≤3:1.

Unlike the aforesaid WO2017/121980 A1, the present invention utilises three synthesis reactors with a single recycle stream. It has surprisingly been found that a three-stage loop can feature a lower heat transfer area reactor in the third stage without requiring an increased circulation around said reactor. The Applicant has found that the methanol-depleted gas mixture to the third stage need not be diluted by an increased circulation, because it has been rendered much less reactive by the previous two reaction stages than the initial make-up gas. Therefore, the exotherm associated with its partial conversion to methanol can be managed by a low heat transfer area reactor without further dilution. This leads to three benefits: (1) High conversion per pass due to the addition of a third reaction stage, therefore high syngas efficiency, (2) Low circulation, therefore low power consumption and small piping, and (3) Low heat transfer area in the third reactor, therefore cheaper reactor internals.

The Applicant has also found that in some cases, and especially at start-up, it is desirable to render the second methanol-depleted gas mixture fed to the third synthesis reactor more reactive. This may be achieved by the following:
(i) a portion of the make-up gas may bypass the first methanol synthesis reactor and be fed to the second methanol synthesis reactor. The portion of the make-up that may by-pass the first synthesis reactor may be $60% vol., preferably ≤40% vol., more preferably 10 to 30% vol;
(ii) a portion of the make-up gas may bypass the first and second methanol synthesis reactors and be fed to the third methanol synthesis reactor. The portion of the MUG that may by-pass the first and second synthesis reactor may be ≤10% vol., preferably ≤5% vol., more preferably 1 to 3% vol;
(iii) a portion of the first methanol-depleted gas mixture may bypass the second methanol synthesis reactor and be fed to the third methanol synthesis reactor. The portion of the first methanol-depleted gas mixture that may by-pass the second synthesis reactor may be ≤20% vol., preferably ≤10% vol., more preferably 4 to 6% vol; or
(iv) a portion of the make-up gas may bypass the first methanol synthesis reactor and be fed to the second methanol synthesis reactor, and a further portion of the make-up gas may bypass the first and second methanol synthesis reactors and be fed to the third methanol synthesis reactor.

In the present invention, the loop recycle gas and make-up gas have a flow rate ratio, or recycle ratio, of ≤3:1. By the term “recycle ratio”, we mean the molar flow ratio of the recycled loop gas to the make-up gas that form the synthesis gas mixture fed to the first synthesis reactor. The recycle ratio to form the first synthesis gas mixture fed to the first synthesis reactor may be ≤2.5, preferably ≤2.0, more preferably ≤1.5, for example in the range 0.7:1 to 1.5:1, preferably 0.8:1 to 1.5:1, more preferably 0.9:1 to 1.5:1. In some arrangements the recycle ratio may be ≤1.4:1, preferably ≤1.3:1.

The first synthesis gas comprises a make-up gas. Make-up gas typically comprises hydrogen, carbon monoxide, and/or carbon dioxide. The make-up gas may be generated by the steam reforming of methane or naphtha using established steam reforming processes, including pre-reforming. However, the present invention is of particular effectiveness in utilising reactive synthesis gases generated by processes including a step of partial oxidation of a hydrocarbon, biomass or carbonaceous feedstock. By “reactive synthesis gases” we mean a synthesis gas comprising hydrogen, carbon monoxide and carbon dioxide in which the ratio (by volume) of carbon monoxide to carbon dioxide is ≥2:1. Such processes include combined reforming in which a first portion of a hydrocarbon feedstock is subjected to steam reforming and a second portion is subjected to autothermal reforming; and from coal or biomass gasification. Alternatively, off-gases from refineries or other chemical processes comprising principally hydrogen and carbon oxides (mainly as carbon monoxide) may also be used. In the present invention, the make-up gas preferably contains carbon monoxide in the range 20 to 35% vol, more preferably 25 to 35% vol.

The use of more reactive synthesis gas leads to smaller catalyst volumes being used, and the greater net heat of reaction gives a heat release per unit volume of catalyst which can be more than double that in a process based on steam reforming alone. Therefore, providing effective cooling of the catalyst becomes more important as the carbon monoxide to carbon dioxide ratio in the synthesis gas increases.

If desired, the make-up gas may be passed through a purification unit comprising one of more purification vessels containing catalysts and/or absorbents that capture contaminants such as sulphur or chloride compounds and prevents them entering the synthesis reactors. This protects the catalysts from poisoning and so extends their lifetime. The purification unit may be installed upstream or, preferably, downstream of a make-up gas compressor.

The make-up gas is combined with the loop recycle gas stream to form the first synthesis gas mixture. The recycle loop stream contains hydrogen and therefore may enhance the methanol formation in the first synthesis reactor where the higher CO-content synthesis gases are used.

The composition of the first synthesis gas mixture at the first synthesis reactor inlet is preferably as follows; 10 to 20 mol % carbon monoxide, 0.5 to 5 mol % carbon dioxide, 65 to 85 mol % hydrogen and the balance one or more inert gases. The pressure of the first synthesis gas at the first synthesis reactor inlet is preferably 50 to 100 bar abs. The temperature of the first synthesis gas at the first synthesis reactor inlet is preferably 200 to 250° C. and the product gas at the outlet preferably 225 to 280° C.

The first methanol-depleted gas mixture is fed to the second synthesis reactor and so may be termed a second feed gas stream. The composition of the first methanol-depleted gas mixture at the second synthesis reactor inlet is preferably as follows; 2 to 10 mol % carbon monoxide, 0.5 to 5 mol % carbon dioxide, 65 to 95 mol % hydrogen and the balance one or more inert gases. The pressure of the first methanol-depleted gas mixture at the second synthesis reactor inlet is preferably 50-100 bar abs. The temperature of the first methanol-depleted gas mixture at the second synthesis reactor inlet is preferably 200 to 250° C. and the product gas at the outlet preferably 225 to 280° C.

The second methanol-depleted gas mixture is fed to the third synthesis reactor and so may be termed a third feed gas stream. The composition of the second methanol-depleted gas mixture at the third synthesis reactor inlet is preferably as follows; 0.2 to 5 mol % carbon monoxide, 0.5 to 5 mol % carbon dioxide, 65 to 95 mol % hydrogen and the balance one or more inert gases. The pressure of the second methanol-depleted gas mixture at the third synthesis reactor inlet is preferably 50 to 100 bar abs. The temperature of the second methanol-depleted gas mixture at the third synthesis reactor inlet is preferably 200 to 250° C. and at the outlet preferably 220 to 290° C.

Upstream of the first, second and third synthesis reactors the feed streams are preferably heated by means of a gas-gas interchanger to provide the desired inlet temperature. A syngas compressor may be provided to increase the pressure of the make-up gas to the desired operating pressure.

The loop recycle gas stream is circulated by means of a circulating compressor to overcome the pressure drop though the synthesis reactors. The circulating compressor is provided to compress either the first synthesis gas mixture, the first methanol-depleted gas mixture or the second methanol depleted gas mixture. The Applicant has found surprisingly that efficiency gains and operating cost advantages exist where the circulating compressor is located between the first and second synthesis reactors, so that the circulating compressor inlet is fed with the first methanol-depleted gas mixture. In preferred embodiment, the circulating compressor is located between the first and second synthesis reactors and a bypass stream from the first methanol-depleted gas mixture is taken downstream of said circulator and fed to the third synthesis reactor. This has the advantage that the by-pass stream is not taken from the make-up gas, but from the first methanol-depleted gas mixture, which has been passed through the first synthesis reactor's catalyst bed, thereby being at least partially purified of any poisons possibly present in the make-up gas. In this way, the third synthesis reactor's catalyst bed is protected against poisoning by at least one upstream synthesis catalyst bed at all times.

In another embodiment, the circulating compressor is located between the first and second synthesis reactors and a portion the make-up gas bypasses the first synthesis reactor and is fed to the suction of said circulating compressor. This configuration can be used with a poison-free make-up gas, which will not cause premature catalyst deactivation in the first and second synthesis reactors.

In another embodiment, the circulating compressor is located between the first and second synthesis reactors and a portion the make-up gas bypasses the first and second synthesis reactors and is fed to the third synthesis reactor. This configuration can be used with a poison-free make-up gas, which will not cause premature catalyst deactivation in the first and third synthesis reactors.

In the present invention, at least part of the third methanol-depleted gas mixture is used to form the recycle gas stream. A purge off-take line may be included at any suitable point in the system to prevent the unwanted build-up of inert gases. For example, a purge may be recovered from the first, second or third methanol depleted gas mixtures. The preferred location for the purge is from the third methanol-depleted gas mixture downstream of the final methanol recovery, because it is the furthest location downstream of the make-up gas addition point. If desired, hydrogen may be recovered from the purge gas and combined with the feed gases to the first, second or third synthesis reactors, for example a recovered hydrogen stream may be added to the make-up gas or first synthesis gas mixture upstream or downstream of a make-up gas compressor. Hydrogen recovery may be performed by pressure-swing absorption or by using suitable membranes.

The process uses first, second and third synthesis reactors in series. If desired, each of the synthesis reactors may have one or more additional synthesis reactors fed in parallel, so that the capacity of the process may be increased.

The first and second synthesis reactors are preferably of a design with a higher heat transfer area relative to the cooled catalyst volume. The heat transfer area can be conveniently characterised by the Volumetric area, or aV. The Volumetric area, or aV, may be defined as the total heat transfer area, A, per cubic metre of cooled catalyst in the reactor. Desirably the first and second synthesis reactors have an aV of ≥50 m²/m³ and more preferably ≥90 m²/m³.

Such converters include those where the catalyst is disposed in a plurality of tubes that are cooled by a heat exchange medium. The first and second synthesis reactors may be the same or different.

The third synthesis reactor has a lower heat transfer area relative to the cooled catalyst volume than the first and second synthesis reactors. For example, the aV may be ≤40 m²/m³. The third synthesis reactor can be of any type meeting this requirement, but high overall conversion of carbon oxides into methanol is associated with low converter exit temperature. There are several converter types that may suitably be used, and these include: (i) converters with gas cooling, such as a tube cooled converter and a gas-cooled converter, and (iii) water-cooled converters with radial flow.

In the present invention, the first and second methanol synthesis reactors have a higher heat transfer area per cubic metre of catalyst than the third synthesis reactor. In some arrangements, it may be desirable to configure the reactors such that the heat transfer area per cubic metre of catalyst of the third synthesis reactor is less than the heat transfer area per cubic metre of catalyst of the second synthesis reactor, which has a lower heat transfer area per cubic metre of catalyst than the first synthesis reactor.

The first methanol-depleted gas mixture typically will have a lower reactivity than the synthesis gas mixture fed to the first synthesis reactor, though not as low as the second methanol-depleted gas mixture. Consequently, it may be desirable for the second reactor to have a volumetric heat transfer area that is lower than the first reactor, though not as low as the third reactor. For example, the first and second reactors may both be axial-flow, steam-raising converters, but where the catalyst-filled tubes of the second synthesis reactor have a larger diameter than the catalyst-filled tubes of the first reactor.

In a preferred arrangement, the first and second synthesis reactors comprise a methanol synthesis catalyst disposed in tubes that are cooled by water under pressure. The third synthesis reactor preferably comprises either a fixed bed of a methanol synthesis catalyst in a radial flow configuration that is cooled in heat exchange with water under pressure or a fixed bed of a methanol synthesis catalyst in a that is cooled in heat exchange with the second methanol-depleted synthesis gas mixture.

Preferably the first and second synthesis reactors are axial-flow, steam-raising converters (aSRC). In such reactors the synthesis gas typically passes axially through vertical, catalyst-containing tubes that are cooled in heat exchange with boiling water under pressure. The catalyst may be provided in pelleted form directly in the tubes or may be provided in one or more cylindrical containers that direct the flow of synthesis gas both radially and axially to enhance heat transfer. An aSRC typically has an aV≥90 m²/m³. Steam raising converters in which the catalyst is present in tubes cooled by boiling water under pressure offer a useful means to remove heat from the catalyst. Axial steam raising converters can be designed to have different volumetric surface areas, e.g. by changing the tube diameter. In this way, a second aSRC can usefully be designed to have a lower volumetric surface area than a first aSRC.

The third synthesis reactor may be a radial-flow steam raising converter, a gas-cooled converter or a tube cooled converter. In each of these, a bed of particulate catalyst is cooled by tubes or plates through which a coolant heat exchange medium passes. The third synthesis reactor could also be a quench reactor in which one or more beds of particulate catalyst are cooled by a synthesis gas mixture injected into the reactor within or between the beds. Such reactors are described, for example, in U.S. Pat. Nos. 3,458,289, 3,475,136 and 4,411,877.

In a radial-flow steam raising converter (rSRC) the synthesis gas typically passes radially (inwards or outwards) through a bed of particulate catalyst which is cooled by a plurality of tubes or plates through which boiling water under pressure is fed as coolant. Such reactors are known and are described for example in U.S. Pat. No. 4,321,234. A rSRC typically has an aV in the range 15-30 m²/m³.

In a tube-cooled converter (TCC), the catalyst bed is cooled by feed synthesis gas passing through open-ended tubes disposed within the bed that discharge the heated gas to the catalyst. A TCC typically has an aV in the range 15-30 m²/m³. As an alternative to a TCC, a gas cooled converter (GCC), may be used to cool the catalyst bed by passing the synthesis gas though tubes in a heat exchanger-type arrangement. A GCC is described for example in the aforesaid

U.S. Pat. No. 5,827,901. The use of a TCC is preferred over the GCC in that it is simpler and cheaper to fabricate due to the use of open topped tubes and the elimination of the upper header and all of the differential expansion problems that the gas cooled converter raises. A TCC therefore has the advantage of low equipment cost and lower outlet temperature, which favours the synthesis reaction equilibrium, but it has a lower heat transfer area than aSRC and higher pressure drop than rSRC.

Alternative converter designs, such as the Linde Variobar converter comprising a bed of methanol synthesis catalyst cooled in heat exchange with boiling water passing through a spiral-wound heat exchanger within the bed, may have an intermediate aV of about 50 m²/m³. Such converters may be used as the third synthesis reactor in combination, for example, with axial-flow steam-raising converters, or may be used as the first and second synthesis reactors in combination with a quench reactor, a tube-cooled converter or a radial-flow steam-raising converter.

The first, second and third synthesis reactors are preferably all cooled by water under pressure. Boiling water under pressure may be provided to the reactors at the same temperature and pressure from a common steam drum. However, the Applicant has found it useful to control the ageing of the catalysts contained in the different reactors using a different water temperature in at least one reactor, during at least part of the catalyst lifetime. For example, at the start-up of the process, the temperatures in the first, second and third synthesis reactors may usefully be the same but as the catalyst ages, it may be advantageous to independently adjust the temperatures of the boiling water such that the catalyst temperatures in each reactor may be optimised. The temperature of water at its boiling point can be controlled by adjusting the pressure of the steam leaving the steam drum, for example by means of a pressure control valve. If each reactor is provided with its own steam drum, the pressure of the steam leaving each steam drum can be controlled independently so that the temperature of the boiling water in each reactor can be controlled independently. In some arrangements where the third synthesis reactor is a radial steam-raising converter (rSRC), a lower boiling water temperature, at least during part of the catalyst lifetime, is advantageous. Accordingly, particularly when the third synthesis reactor is a radial steam-raising converter it is particularly desirable to operate it with a lower boiling water temperature at least during part of the catalyst lifetime. This increases the temperature difference between the reacting gas and the boiling water, which is the heat transfer driving force. Accordingly, the steam leaving the steam drum of the third reactor can be set to a lower pressure than the steam leaving the steam drums of the first two reactors. Furthermore, in arrangements where the catalyst-filled tubes of the second synthesis reactor have a larger diameter than the catalyst-filled tubes of the first synthesis reactor, it may be desirable to run the second reactor with a lower boiling water temperature than the first reactor, though not as low as the third reactor. In this case, the steam leaving the steam drum feeding the second reactor can be set to a lower pressure than the steam leaving the steam drum feeding the first reactor, though not as low as the pressure of the steam leaving the steam drum feeding the third reactor. In configurations where the catalyst-filled tubes of the second synthesis reactor have the same diameter as the catalyst-filled tubes of the first reactor, for ease of operation, it may be desirable to run the first and second reactor with the same boiling water temperature. In this case, the first and second synthesis reactors may share the same steam drum. By controlling the pressure of the steam leaving the common steam drum, the temperature of the boiling water in the first and second synthesis reactor can be set to the same value and controlled simultaneously.

The methanol synthesis catalysts are preferably copper-containing methanol synthesis catalysts, in particular the methanol synthesis catalyst in the first and second synthesis reactors is a particulate copper/zinc oxide/alumina catalyst. Particularly suitable catalysts are Mg-doped copper/zinc oxide/alumina catalysts as described in U.S. Pat. No. 4,788,175 and Si-doped copper/zinc oxide/alumina catalysts as described in WO/2020/212681 A1. The same or different methanol synthesis catalysts may be used in the first, second and third synthesis reactors.

Methanol synthesis may be effected in the first and second synthesis reactors at elevated temperature and pressure, for example pressures in the range 20 to 120 bar abs and temperatures in the range 130° C. to 350° C.

The product gas streams from the first, second and third synthesis reactors may be cooled in one or more stages of heat exchange, e.g. with water or air cooling, to condense methanol therefrom, which may suitably be recovered using gas-liquid separators. The cooling may be performed to fully or partially condense the methanol from the first, second and third product gas streams. Preferably essentially all of the methanol is condensed from the third product gas stream. The recovered liquid methanol streams may be processed separately or may be combined and passed for further processing. The methanol contains water, dissolved gases and may contain small amounts of organic contaminants. Therefore, the further processing may comprise treatment in a stabilisation unit to de-gas the methanol and produce a stabilised methanol product suitable for conversion into olefins. The further processing may also comprise purification in a purification unit comprising one or more, preferably two or three, stages of distillation to separate out water and contaminants and produce a purified methanol product.

The proportion of the methanol made in the first, second and third synthesis reactors may be adjusted by a by-pass around the second synthesis reactor. Preferably the methanol production in the first reactor is greater than the methanol production in each of the second and third reactors. Preferably the methanol production in the second reactor is greater than the methanol production in the third reactor.

The invention will be further described by reference to the figures in which;

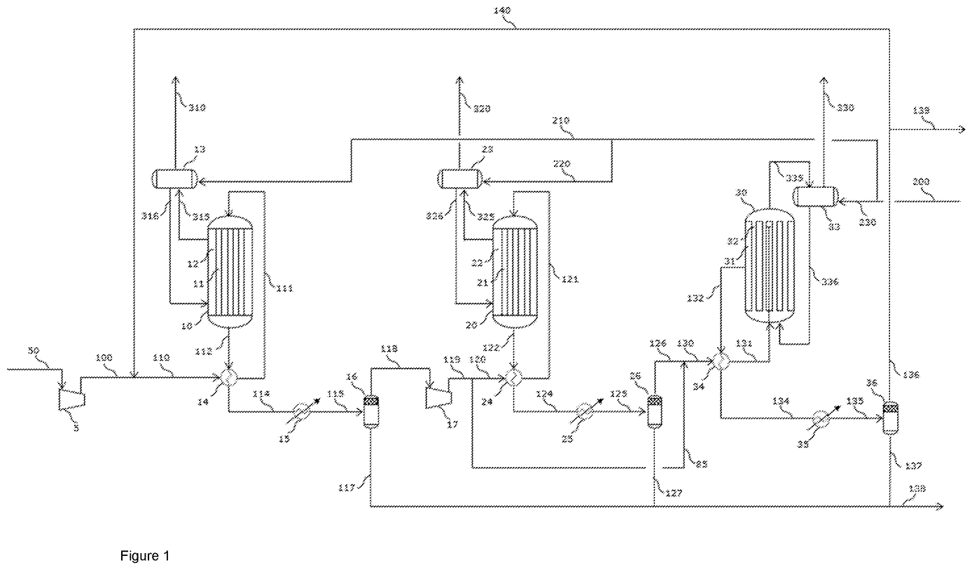

FIG. 1 depicts a process according to an embodiment of the present invention utilising two aSRC reactors and a rSRC reactor, with a bypass of unreacted gas around the second synthesis reactor;

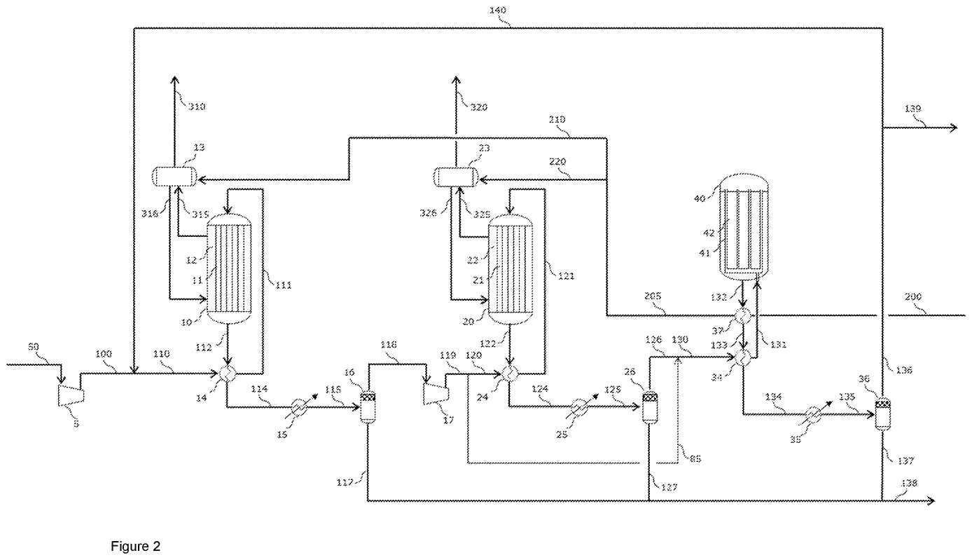

FIG. 2 depicts a process according to an embodiment of the present invention utilising two aSRC reactors and a TCC reactor with a bypass of unreacted gas around the second synthesis reactor; and

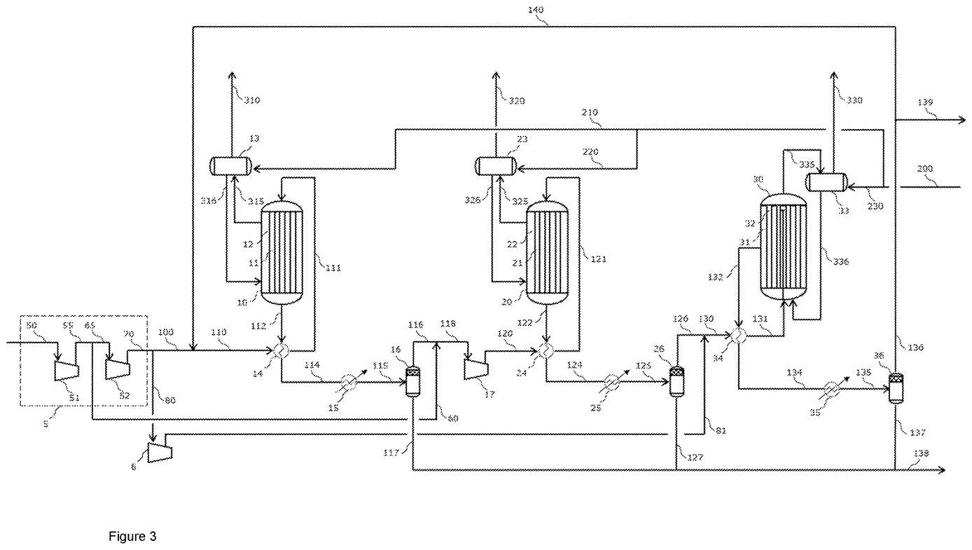

FIG. 3 depicts a process according to a further embodiment of the present invention utilising two aSRC reactors and a rSRC reactor, with bypass of make-up gas around the first synthesis reactor and a further bypass of make-up gas around the first and second synthesis reactors.

It will be understood by those skilled in the art that the drawings are diagrammatic and that further items of equipment such as feedstock drums, pumps, vacuum pumps, compressors, gas recycling compressors, temperature sensors, pressure sensors, pressure relief valves, control valves, flow controllers, level controllers, holding tanks, storage tanks and the like may be required in a commercial plant. Provision of such ancillary equipment forms no part of the present invention and is in accordance with conventional chemical engineering practice.

In FIG. 1, a make-up gas in line 50 comprising hydrogen, carbon monoxide and carbon dioxide is compressed to the shell side inlet pressure of gas-gas interchanger 14 in make-up syngas compressor 5. The compressed make-up gas stream 100 is combined with a recycle stream 140 to form first feed gas stream 110, which is fed to the shell side of gas-gas interchanger 14 where it is heated in indirect heat exchange with a first product gas stream 112. The heated first feed gas stream is fed by line 111 to the inlet of an axial steam-raising converter 10, containing catalyst-filled tubes 11 through which the synthesis gas mixture is passed. The catalyst is a particulate copper/zinc oxide/alumina catalyst. Boiling water under pressure is fed to the shell side 12 of the reactor through downcomer 316 and a mixture of boiling water and steam is withdrawn and supplied to a steam drum 13 through riser 315. The methanol synthesis reaction takes place as the synthesis gas passes axially through the catalyst-filled tubes 11 to form a first product gas stream containing methanol vapour. A first product gas stream is recovered from the outlet of the first synthesis reactor 10 and fed via line 112 to the tube side of gas-gas interchanger 14 where it is partially cooled. The partially cooled gas is fed via line 114 to one or more further stages of heat exchange 15 to condense methanol therefrom. The resulting gas-liquid mixture is passed via line 115 to a gas-liquid separator 16 and liquid methanol is recovered via line 117.

A first methanol-depleted gas mixture comprising unreacted hydrogen and carbon oxides is recovered from the separator 16 and fed via line 118 to circulator 17 where it is compressed to the shell side inlet pressure of gas-gas interchanger 24 and fed to line 119. Compressed stream 119 is separated into an optional bypass stream 85 and a second feed gas stream 120.

The second feed gas stream 120 is fed to the shell side of gas-gas interchanger 24 where it is heated in indirect heat exchange with a second product gas stream 122. The heated second feed gas stream is fed by line 121 to the inlet of an axial steam-raising converter 20, containing catalyst-filled tubes 21 through which the synthesis gas mixture is passed. The catalyst is a particulate copper/zinc oxide/alumina catalyst. The boiling water under pressure is fed to the shell side 22 of the reactor through downcomer 326 and a mixture of boiling water and steam is withdrawn and supplied to a steam drum 23 through riser 325. The methanol synthesis reaction takes place as the synthesis gas passes axially through the catalyst-filled tubes 21 to form a second product gas stream containing methanol vapour. The second product gas stream is recovered from the outlet of the second synthesis reactor 20 and fed via line 122 to the tube side of gas-gas interchanger 24 where it is partially cooled. The partially cooled gas is fed via line 124 to one or more further stages of heat exchange 25 to condense methanol therefrom. The resulting gas-liquid mixture is passed via line 125 to a gas-liquid separator 26 and liquid methanol is recovered via line 127. A second methanol-depleted gas mixture comprising unreacted hydrogen and carbon oxides is recovered from the separator 26 and fed to line 126 and mixed with the optional bypass stream 85 to form third feed gas stream 130.

Third feed gas stream 130 is fed to the shell side of gas-gas interchanger 34 where it is heated in indirect heat exchange with a third product gas stream 132. The heated third feed gas stream is fed by line 131 to the inlet of a radial steam-raising converter 30, containing a bed of methanol synthesis catalyst 31, containing a plurality of heat exchange tubes 32 though which boiling water under pressure is passed as coolant. Whereas tubes are depicted, alternative heat exchange devices such as plates through which the coolant may be passed, may also be used. The boiling water under pressure is fed to the tube side 32 of the reactor 30 through downcomer 336 and a mixture of boiling water and steam is withdrawn and supplied to a steam drum 33 through riser 335. The methanol synthesis reaction takes place as the synthesis gas passes radially through the bed of catalyst 31 to form a third product gas stream containing methanol vapour. The third product gas stream is recovered from the outlet of the third synthesis reactor 30 and fed via line 132 to the tube side of gas-gas interchanger 34 where it is partially cooled. The partially cooled gas is fed via line 134 to one or more further stages of heat exchange 35 to condense methanol therefrom. The resulting gas-liquid mixture is passed via line 135 to a gas-liquid separator 36 and liquid methanol is recovered via line 137. A final methanol-depleted gas mixture comprising unreacted hydrogen and carbon oxides is recovered from the separator 36 and fed by line 136 to a purge off-take line 139, which removes a portion of the gas to reduce the build-up of inert gases.

The remaining final methanol-depleted gas mixture from line 136 forms the recycle stream 140. The crude methanol streams 117, 127 and 137 are combined and sent by line 138 for further processing such as one or more stages of distillation to produce a purified methanol product. A boiler feed water stream 200 is divided into streams 210, 220 and 230, which are fed to steam drums 13, 23 and 33 respectively.

FIG. 2 depicts the same processes as FIG. 1 but replaces the radial steam raising converter with a tube-cooled converter 40 in which the catalyst bed is cooled in heat exchange with the third feed gas stream (the second methanol-depleted synthesis gas 126 plus optional bypass stream 85). Thus, the third feed gas stream is fed from heat exchanger 34 via line 131 to the bottom of a tube cooled converter 40 and passed upwards through a plurality of tubes 41 disposed within the catalyst bed 42. The gas is heated as it passes upwards through tubes. The heated gas exits the tubes within the reactor above the bed and then passes down through the bed where it reacts to form a gas mixture containing methanol vapour. The product gas is collected and fed via line 132 to heat exchanger 34 where it is cooled to condense methanol. In this arrangement, it is desirable to first recover heat from the product stream 132 in heat exchange with the boiler feed water 200 in a heat exchanger 37 that feeds a stream of heated water via lines 210 and 220 to the steam drums 13 and 23.

FIG. 3 depicts the same processes as FIG. 1 but bypass line 85 is replaced by first bypass line 60 and second bypass line 80. Additionally, the make-up syngas compressor 5 is a two-stage machine comprising low pressure stage 51 and high pressure stage 52, and an additional bypass syngas compressor 6 is provided to compress the second bypass stream 80. Thus, the make-up syngas in line 50 is compressed to the suction pressure of circulator 17 in the low-pressure stage 51 of the two-stage make-up gas compressor 5 and fed to line 55. The partially compressed syngas stream 55 is divided into the first bypass stream 60 and a residual partially compressed syngas stream 65. The residual partially compressed syngas stream 65 is further compressed to the shell side inlet pressure of gas-gas interchanger 14 in the high-pressure stage 52 of the two-stage make-up gas compressor 5 and fed to line 70. The compressed residual syngas stream 70 is divided into the second bypass stream 80 and a compressed make-up syngas stream 100. The compressed make-up syngas stream 100 is combined with a recycle stream 140 to form first feed gas stream 110.

The first bypass stream 60 is combined with the first methanol-depleted gas mixture 116.

The second bypass stream 80 is compressed to the shell side inlet pressure of gas-gas interchanger 34 in the additional bypass syngas compressor 6 and fed via line 81 to be combined with the second methanol-depleted gas mixture 126.

The rest of the scheme is then the same as in FIG. 1. In FIG. 3 both first and second bypass streams 60 and 80 are shown. Although such a configuration is possible where bypass streams 60 and 80 are both present at the same time, separate embodiments where either the first bypass stream 60 is present or the second bypass stream 80 is present may also be used. If only bypass stream 60 is present, additional syngas compressor 6 is not required.

The Invention is further illustrated by reference to the following Examples.

### Example 1

A computer model of a process based upon the flowsheet depicted in FIG. 1. The compositions, temperatures and pressures of the streams depicted in FIG. 1 are set out in the following tables.

| Stream Number | | 50 | 85 | 100 | 110 | 111 | 112 | 114 | 115 | 117 |
|---|---|---|---|---|---|---|---|---|---|---|
| Molar Flow | kNm³/h | 780.7 | 62.9 | 780.7 | 1795.6 | 1795.6 | 1439.2 | 1439.2 | 1439.2 | 181.0 |
| Mass Flow | t/h | 379.2 | 26.1 | 379.2 | 777.9 | 777.9 | 777.9 | 777.9 | 777.9 | 255.6 |
| Temperature | ° C. | 45.0 | 69.0 | 73.1 | 57.4 | 211.0 | 235.4 | 113.8 | 45.0 | 45.0 |
| Pressure | bara | 61.5 | 87.6 | 77.6 | 77.6 | 77.2 | 74.8 | 73.7 | 72.3 | 72.2 |

| Molar | | | | | | | | | | |
|---|---|---|---|---|---|---|---|---|---|---|
| Composition | Unit | | | | | | | | | |
| Water | mol % | 0.2 | 0.0 | 0.2 | 0.1 | 0.1 | 0.3 | 0.3 | 0.3 | 2.4 |
| Hydrogen | mol % | 67.4 | 75.0 | 67.4 | 72.6 | 72.6 | 65.6 | 65.6 | 65.6 | 0.4 |
| Carbon Monoxide | mol % | 29.2 | 4.1 | 29.2 | 12.7 | 12.7 | 3.6 | 3.6 | 3.6 | 0.1 |
| Carbon Dioxide | mol % | 2.4 | 1.4 | 2.4 | 1.1 | 1.1 | 1.3 | 1.3 | 1.3 | 0.4 |
| Methanol | mol % | 0.0 | 0.7 | 0.0 | 0.3 | 0.3 | 12.7 | 12.7 | 12.7 | 96.1 |
| Inerts | mol % | 0.8 | 18.7 | 0.8 | 13.2 | 13.2 | 16.4 | 16.4 | 16.4 | 0.5 |

| Stream Number | | 118 | 119 | 120 | 121 | 122 | 124 | 125 | 126 | 127 |
|---|---|---|---|---|---|---|---|---|---|---|
| Molar Flow | kNm³/h | 1258.2 | 1258.2 | 1195.3 | 1195.3 | 1083.2 | 1083.2 | 1083.2 | 1013.6 | 69.5 |
| Mass Flow | t/h | 522.2 | 522.2 | 496.1 | 496.1 | 496.1 | 496.1 | 496.1 | 403.2 | 92.9 |
| Temperature | ° C. | 45.0 | 69.0 | 69.0 | 209.0 | 228.3 | 103.4 | 45.0 | 45.0 | 45.0 |
| Pressure | bara | 72.2 | 87.6 | 87.6 | 87.1 | 84.7 | 83.9 | 82.5 | 82.4 | 82.4 |

| Molar | | | | | | | | | | |
|---|---|---|---|---|---|---|---|---|---|---|
| Composition | Unit | | | | | | | | | |
| Water | mol % | 0.0 | 0.0 | 0.0 | 0.0 | 1.0 | 1.0 | 1.0 | 0.0 | 14.4 |
| Hydrogen | mol % | 75.0 | 75.0 | 75.0 | 75.0 | 71.5 | 71.5 | 71.5 | 76.4 | 0.3 |
| Carbon Monoxide | mol % | 4.1 | 4.1 | 4.1 | 4.1 | 0.4 | 0.4 | 0.4 | 0.4 | 0.0 |
| Carbon Dioxide | mol % | 1.4 | 1.4 | 1.4 | 1.4 | 0.6 | 0.6 | 0.6 | 0.6 | 0.2 |
| Methanol | mol % | 0.7 | 0.7 | 0.7 | 0.7 | 5.9 | 5.9 | 5.9 | 0.5 | 84.6 |
| Inerts | mol % | 18.7 | 18.7 | 18.7 | 18.7 | 20.7 | 20.7 | 20.7 | 22.0 | 0.5 |

| Stream Number | | 130 | 131 | 132 | 134 | 135 | 136 | 137 | 138 | 139 |
|---|---|---|---|---|---|---|---|---|---|---|
| Molar Flow | kNm³/h | 1076.6 | 1076.6 | 1053.8 | 1053.8 | 1053.8 | 1035.6 | 18.2 | 268.8 | 20.6 |
| Mass Flow | t/h | 429.3 | 429.3 | 429.3 | 429.3 | 429.3 | 406.8 | 22.5 | 371.0 | 8.1 |
| Temperature | ° C. | 46.4 | 210.0 | 229.6 | 70.0 | 45.0 | 45.0 | 45.0 | 45.0 | 45.0 |
| Pressure | bara | 82.1 | 81.3 | 80.6 | 79.5 | 78.1 | 78.0 | 78.0 | 72.2 | 77.7 |

| Molar | | | | | | | | | | |
|---|---|---|---|---|---|---|---|---|---|---|
| Composition | Unit | | | | | | | | | |
| Water | mol % | 0.0 | 0.0 | 0.6 | 0.6 | 0.6 | 0.1 | 31.0 | 7.4 | 0.1 |
| Hydrogen | mol % | 76.3 | 76.3 | 75.2 | 75.2 | 75.2 | 76.5 | 0.2 | 0.4 | 76.5 |
| Carbon Monoxide | mol % | 0.6 | 0.6 | 0.1 | 0.1 | 0.1 | 0.1 | 0.0 | 0.1 | 0.1 |
| Carbon Dioxide | mol % | 0.7 | 0.7 | 0.1 | 0.1 | 0.1 | 0.1 | 0.0 | 0.3 | 0.1 |
| Methanol | mol % | 0.6 | 0.6 | 1.6 | 1.6 | 1.6 | 0.5 | 68.5 | 91.3 | 0.5 |
| Inerts | mol % | 21.8 | 21.8 | 22.3 | 22.3 | 22.3 | 22.7 | 0.3 | 0.5 | 22.7 |

| Stream Number | | 140 | 200 | 210 | 220 | 230 | 310 | 320 | 330 |
|---|---|---|---|---|---|---|---|---|---|
| Molar Flow | kNm³/h | 1014.9 | 495.2 | 381.9 | 104.6 | 8.7 | 374.4 | 102.5 | 8.6 |
| Mass Flow | t/h | 398.7 | 398.0 | 307.0 | 84.1 | 7.0 | 300.9 | 82.4 | 6.9 |
| Temperature | ° C. | 45.0 | 104.0 | 104.6 | 104.6 | 104.6 | 228.0 | 228.0 | 228.0 |
| Pressure | bara | 77.6 | 58.9 | 27.5 | 27.5 | 27.5 | 27.0 | 27.0 | 27.0 |

| Molar | | | | | | | | | |
|---|---|---|---|---|---|---|---|---|---|
| Composition | Unit | | | | | | | | |
| Water | mol % | 0.1 | 100.0 | 100.0 | 100.0 | 100.0 | 100.0 | 100.0 | 100.0 |
| Hydrogen | mol % | 76.5 | 0.0 | 0.0 | 0.0 | 0.0 | 0.0 | 0.0 | 0.0 |
| Carbon Monoxide | mol % | 0.1 | 0.0 | 0.0 | 0.0 | 0.0 | 0.0 | 0.0 | 0.0 |
| Carbon Dioxide | mol % | 0.1 | 0.0 | 0.0 | 0.0 | 0.0 | 0.0 | 0.0 | 0.0 |
| Methanol | mol % | 0.5 | 0.0 | 0.0 | 0.0 | 0.0 | 0.0 | 0.0 | 0.0 |
| Inerts | mol % | 22.7 | 0.0 | 0.0 | 0.0 | 0.0 | 0.0 | 0.0 | 0.0 |

The bypass stream 85 improves the performance of the third synthesis reactor. As the catalyst ages during use, its activity reduces, and the by-pass becomes less useful. The process may therefore also be operated without the by-pass, especially at end of life (EOL) of the methanol synthesis catalyst.

### Comparative Examples 1 and 2

Comparative example 1 comprises a two-stage loop as disclosed in WO2017121980 (A1), FIG. 1.

Comparative Example 2 is the same as the invention depicted in FIG. 1, but the radial steam-raising reactor 30 has been replaced by an axial steam raising reactor (of the same type as reactors 10 and 20).

The following table compares the relevant performance indicators of the Invention depicted in FIG. 1 (Example 1) compared with Comparative Examples 1 and 2.

| | | | | aV | aV |
|---|---|---|---|---|---|
| | Methanol | EOL Syngas | aV | Second | Third |
| | Production | consumption | First Stage | Stage | Stage |
| | t/h | kNm³/h | m²/m³ | m²/m³ | m²/m³ |
| Example 1 | 350 | 766 | 99 | 99 | 24 |
| Comparative | 350 | 769 | 99 | 24 | — |
| Example 1 | | | | | |
| Comparative | 350 | 766 | 99 | 99 | 99 |
| Example 2 | | | | | |

The results indicate that:
For the same methanol production, Comparative Example 1 has a higher EOL syngas consumption (769 vs 766 kNm³/h)
For the same methanol production, Comparative Example 2 has the same EOL syngas consumption (766 kNm³/h), but the aV of the third stage reactor is 99 versus 24 m²/m³, so, requires a more complex and expensive converter.

### Comparative Examples 3 and 4

Comparative Example 3 is the same as the invention depicted in FIG. 1, but with the circulator 17 located upstream of gas-gas interchanger 34.

Comparative Example 4 is the same as the invention depicted FIG. 1, but with circulator 17 located upstream of gas-gas interchanger 14.

The following table compares the relevant performance indicators of the Invention depicted FIG. 1 (Example 1) to Comparative Examples 3 and 4.

| | Boiling water | First Reactor | Second Reactor | Third Reactor |
|---|---|---|---|---|
| | temperature at | peak | peak | peak |
| | BOL | temperature at | temperature at | temperature at |
| | (all reactors) | BOL | BOL | BOL |
| | ° C. | ° C. | ° C. | ° C. |
| Example 1 | 228 | 256 | 250 | 234 |
| Comparative | 228 | 261 | 246 | 234 |
| Example 3 | | | | |
| Comparative | 228 | 267 | 247 | 233 |
| Example 4 | | | | |

The results indicate that at beginning of life (BOL) of the methanol synthesis catalyst for a given boiling water temperature (228° C.), the peak temperature of the first reactor is the highest of the three reactors in all cases. Therefore, the catalyst in the first reactor is expected to experience the fastest thermal sintering of the three. Advantageously, the Invention provides the lowest peak temperature in the first reactor (256 vs. 261 and 267° C.). The peak temperature in the first reactor is important because, in all examples, the first reactor makes more methanol than the other two combined, as can be seen in the table below.

| | | Methanol Production | |
|---|---|---|---|
| | Methanol Production | from Second | Methanol Production |
| | from First Reactor | Reactor | from Third Reactor |
| | t/h | t/h | t/h |
| Example 1 | 249 | 84 | 18 |
| Comparative | 264 | 69 | 17 |
| Example 3 | | | |
| Comparative | 280 | 56 | 15 |
| Example 4 | | | |

Therefore, the overall methanol productivity will benefit the most by keeping the first converter peak temperature as low as possible, because this contributes to slowing down the thermal degradation of the catalyst in the first synthesis reactor.

The benefits of operating the process of the invention are as follows:
1. Using three reaction stages provides higher methanol production, therefore high syngas efficiency with the low recycle ratio because the reaction equilibrium is further shifted by condensing three times instead of twice, thus giving a higher conversion per pass.
2. Using a reactor for the third stage with a lower heat transfer area per cubic metre of catalyst means less steel for the internals, therefore cheaper construction. It also enables reactor designs, such as radial-flow steam raising reactors and tube cooled reactors, with the catalyst on the shell side, thus with more catalyst per cubic meter of reactor. This has the potential to decrease the need for a parallel reactor for high-capacity plants.
3. The location of the circulator between the first and second synthesis reactors means that the first synthesis reactor has the lowest inlet pressure of the three, which compensates for the high reactivity of the first feed gas stream. This gives the lowest peak temperature in the first reactor for a given temperature of the boiling water. This is particularly advantageous at beginning of life (start-up), in that the peak temperature of the first synthesis reactor can be limited without the need to excessively decrease the temperature of the steam raised. This contributes to slowing down the thermal degradation of the catalyst in the first synthesis reactor. Any other location of the circulator would increase the inlet pressure to the first synthesist reactor with respect to at least one of the other two, which in turn would increase its peak temperature for a given temperature of the boiling water.

1. A process for the synthesis of methanol comprising the steps of:
(i) passing a first synthesis gas mixture comprising a make-up gas and a loop recycle gas stream through a first synthesis reactor containing a cooled methanol synthesis catalyst to form a first product gas stream,
(ii) recovering methanol from the first product gas stream thereby forming a first methanol-depleted gas mixture,
(iii) passing at least a portion of the first methanol-depleted gas mixture through a second synthesis reactor containing a cooled methanol synthesis catalyst to form a second product gas stream,
(iv) recovering methanol from the second product gas stream thereby forming a second methanol-depleted gas mixture,
(v) passing the second methanol-depleted gas mixture through a third synthesis reactor containing a cooled methanol synthesis catalyst to form a third product gas stream,
(vi) recovering methanol from the third product gas stream thereby forming a third methanol-depleted gas mixture; and
(vii) feeding a portion of the third methanol-depleted gas mixture to the first methanol synthesis reactor as the loop recycle gas stream,
wherein the first and second synthesis reactors have a higher heat transfer area per cubic metre of catalyst than the third synthesis reactor, a circulating compressor is provided to compress either the first synthesis gas mixture, the first methanol-depleted gas mixture or the second methanol depleted gas mixture, and the loop recycle gas and make-up gas have a molar flow rate ratio of ≤3:1.

2. The process according to claim 1, wherein the make-up gas contains carbon monoxide in the range 20-35% vol.

3. The process according to claim 1, wherein the circulating compressor is provided to compress the first methanol-depleted gas mixture.

4. The process according to claim 1, wherein the flow rate ratio of the loop recycle gas to make-up gas is ≤2.5.

5. The process according to claim 1, wherein the first and second synthesis reactors comprise a methanol synthesis catalyst disposed in tubes that are cooled by water under pressure.

6. The process according to claim 5, wherein the third synthesis reactor comprises a fixed bed of a methanol synthesis catalyst that is cooled in heat exchange with either water under pressure or a synthesis gas mixture.

7. The process according to claim 6, wherein each synthesis reactor is provided with its own steam drum and the pressure of the steam leaving each steam drum is controlled independently such that the temperature of the water in each reactor is controlled independently, or wherein the first and second synthesis reactors share a common steam drum and the temperature of the boiling water in the first and second synthesis reactors is set to the same value and controlled simultaneously.

8. The process according to claim 7, wherein the heat transfer area per cubic metre of catalyst of the third synthesis reactor is less than the heat transfer area per cubic metre of catalyst of the second synthesis reactor, which has a lower heat transfer area per cubic metre of catalyst than the first synthesis reactor.

9. The process according to claim 8, wherein the first and second synthesis reactors are both axial-flow steam-raising converters containing catalyst filled tubes cooled by water under pressure, where the catalyst-filled tubes of the second synthesis reactor have a larger diameter than the catalyst-filled tubes of the first reactor.

10. The process according to claim 5, wherein the third synthesis reactor is a radial steam-raising converter cooled by water under pressure at a lower temperature than the water under pressure used to cool the first and/or second synthesis reactors.

11. The process according to claim 1, wherein at least at the start-up of the process a portion of the make-up gas is bypassed around the first synthesis reactor and fed to the second synthesis reactor.

12. The process according to claim 1, wherein at least at the start-up of the process a portion of the make-up gas is bypassed around the first and second synthesis reactors and fed to the third synthesis reactor.

13. The process according to claim 1, wherein at least at the start-up of the process a portion of the first methanol-depleted gas mixture is bypassed around the second synthesis reactor and fed to the third synthesis reactor.

14. The process according to claim 13, wherein the amount of by-pass is ≤20% vol of the first methanol-depleted gas mixture.

15. The process according to claim 1, wherein a purge gas stream is recovered from the first, second or third methanol depleted gas mixture, hydrogen is recovered from the purge stream and fed to the first, second or third synthesis reactors.

16. A process according to claim 1, wherein the product gas streams from the first, second and third synthesis reactors are cooled in one or more stages of heat exchange to condense methanol therefrom, and the condensed methanol is fed to a stabilisation unit to produce a stabilised methanol product suitable for conversion into olefins or is fed to a purification unit comprising one or more distillation stages to produce a purified methanol product.

17. The process according to claim 1, wherein the flow rate ratio of the loop recycle gas to make-up gas is ≤2.0.

18. The process according to claim 1, wherein the flow rate ratio of the loop recycle gas to make-up gas is ≤1.5.

19. The process according to claim 1, wherein the first and second synthesis reactors are both axial flow steam-raising converters.

20. The process according to claim 5, wherein the third synthesis reactor is selected from a radial flow steam-raising converter, a tube-cooled converter, and a gas-cooled converter.
